(19) United States
(12) Patent Application Publication
(10) Pub. No.: US 2016/0256098 A1
(43) Pub. Date: Sep. 8, 2016

(54) METHOD FOR DIAGNOSING BIRTHMARKS ON THE SKIN

(71) Applicant: KTG SP. Z O.O, Warszawa (PL)

(72) Inventor: MARCINÓW GROCHOWINA, RZESZÓW (PL)

(73) Assignee: KTG Sp. z o.o, Warszawa (PL)

(21) Appl. No.: 14/435,503

(22) PCT Filed: Jul. 24, 2014
(86) PCT No.: PCT/PL2014/000086

§ 371 Date: Apr. 14, 2015

(30) Foreign Application Priority Data
Jun. 28, 2014 (PL) P.408689

(51) Int. Cl. A61B 5/00

(52) U.S. Cl. CPC A61B 5/444; A61B 5/0077; A61B 5/4842; A61B 5/7246; A61B 5/0013; A61B 2576/02

(57) ABSTRACT

A method of diagnosing birthmarks on the skin with the use of the microprocessor diagnostic system characterized in that the image of skin area recorded by a camera (1) is transmitted to the microprocessor diagnostic system (2), and implemented in its interior algorithms executed by the microprocessor perform image analysis in order to obtain diagnostic information, wherein at first area subjected to testing is typed (3) within which the birthmarks are detected (4), then the coordinates of the birthmarks are determined and they are counted (5) and compared (6) with previous records of the same skin area stored in the database (8), giving the result (7) constituting the material, which is determining the growth of the amount of birthmarks and its dynamics on a given area of the skin.

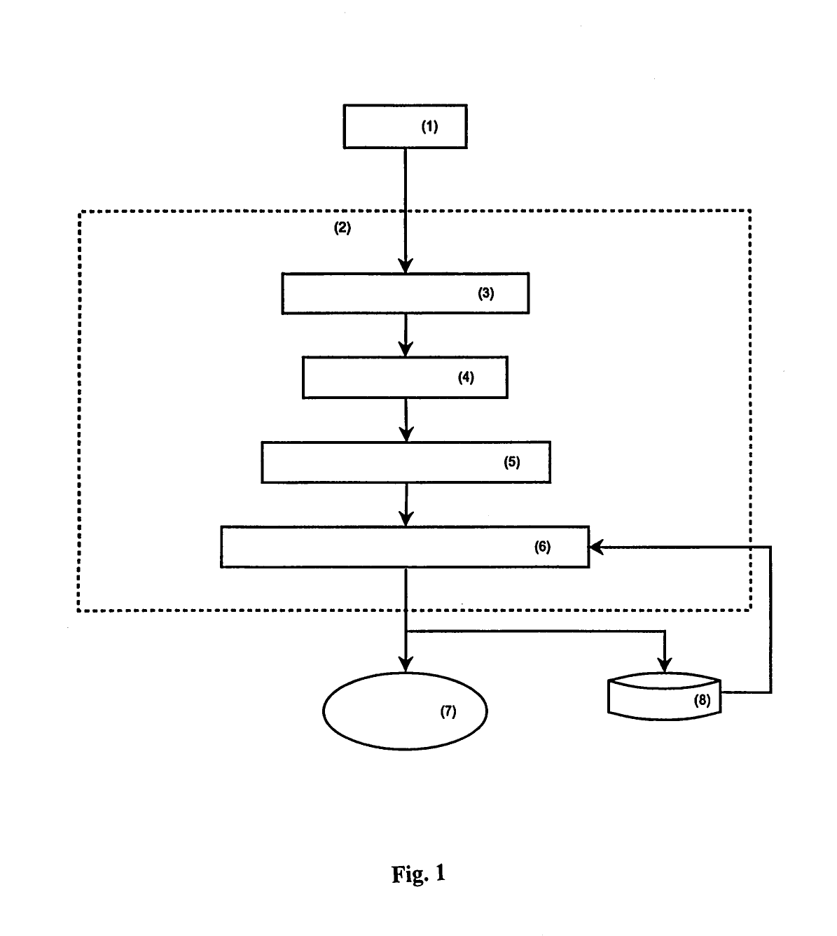

The present invention is a method of diagnosing birthmarks on the skin, which consists in non-invasive typing of an area, determination of the birthmarks and their coordinates, counting and analysis.

Method of detecting melanoma marks intended for its non-invasive diagnosis is known from the patent description PL175972. The known method consists in that the birthmark and its vicinity is irradiated with ultraviolet light having a wavelength of 350 to 380 nm, preferably 360 to 370 nm and with the help of the digital image recording system the intensity of the birthmark autofluorescence and its surrounding in the band of 460 to 490 nm, preferably 470 to 480 nm is analysed at the same time, wherein as a criterion for the diagnosis of melanoma is determined the ration of the maximum autofluorescence intensity in the surrounding of melanoma to its minimum intensity in the area of the birthmark.

A method for the detection of melanoma cells in paraffin formulations is known also from patent application description P.386772. Paraffin formulation of human skin pigmentation change preserved in formalin is illuminated with blue light having a wavelength in the range of 410 to 450 nm in order to excite the fluorescence, which digital image is recorded while keeping the photometric requirements in the band from 450 to 550 nm. The resulting images are analysed in the centre of a change. The average fluorescence intensity values are calculated in the manually or automatically designated areas on the image, based on which, with the use of a non-linear classifier, the result and the indicators of its reliability are determined.

Suddenly was found, that using a microprocessor diagnostic system the test result is obtained in a way, which is enabling the detection and analysis of the state of tested birthmarks by comparison with the earlier result without using invasive methods of diagnosis.

The essence of the invention lies in that the image of the skin area recorded by a camera is transmitted to the microprocessor diagnostic system and algorithms implemented in its inside executed by a microprocessor carry out the image analysis to obtain diagnostic information, wherein at first the area subjected to testing is typed, within which the birthmarks are detected, then the coordinates of the birthmarks are determined and they are counted, as a result a material is obtained, which is compared with previous earlier records of the same skin area-stored in the database, whereas as a result of comparison a material is obtained, which is determining the growth of the amount of birthmarks and its dynamics on a given area of the skin.

The present invention was demonstrated in the embodiment in the drawing in a block diagram form.

Microprocessor diagnostic system 2 equipped with a camera 1 allows predicting the test area 3, the detection of the birthmarks, determining their coordinates and counting 5, and afterwards compares with the previous test 6 if it was already in the database 8, and gives a test result 7.

1. A method of diagnosing birthmarks on the skin with the use of the microprocessor diagnostic system characterized in that the image of skin area recorded by a camera (1) is transmitted to the microprocessor diagnostic system (2), and implemented in its interior algorithms executed by the microprocessor perform an image analysis in order to obtain diagnostic information, wherein at first the area subjected to testing is typed (3) within which the birthmarks are detected (4), then the coordinates of the birthmarks are determined and they are counted (5) and compared (6) with previous records of the same skin area stored in the database (8), giving the result (7) constituting the material, which is determining the growth of the amount of birthmarks and its dynamics ona given area of the skin.
